(19) United States
(12) Patent Application Publication
(10) Pub. No.: US 2004/0068168 A1
(43) Pub. Date: Apr. 8, 2004

(54) Coordinative resonance detection by a coordinated feedback system

(76) Inventor: Louis A. Lala, West Carrolton, OH (US)

Correspondence Address: A PATENT LAWYER CORP, PC, R WILLIAM GRAHAM, 22 S ST CLAIR ST, DAYTON, OH, 45402, US

(21) Appl. No.: 10/243,497

(22) Filed: Sep. 13, 2002

(51) Int. Cl. A61B 5/05

(52) U.S. Cl. 600/407; 600/382; 348/77

(57) ABSTRACT

A resonance system is provided for use on a subject having a biological pathology. The system includes a wave generator operably disposed adjacent a subject for generating a wave (which can be an electric wave) at a predetermined frequency, a resonant frequency sensor and generator operably disposed adjacent the subject for sensing a resonant frequency of a predetermined area of the subject in response to the predetermined frequency and generating a resonant frequency signal in response to said sensed resonant frequency, and a device (which is preferably computer based) operably associated with the sensor for receiving the resonant frequency signal and manipulating the resonant frequency signal in a manner to be displayed. A method of detecting a resonant frequency in a subject having a biological pathology and treating the pathology using the sensed resonant frequency are also provided.

### BACKGROUND OF THE INVENTION

1. Field of the Invention

The present invention relates to an ability to detect pathology in a human body. In addition, the invention is directed to a device and method for applying a resonant frequency to treat a predetermined pathology in manner to eradicate the same.

2. Prior art

Some time ago (early 1930s), significant studies were conducted by Dr. Royal Raymond Rife wherein the pathology of live tissue was examined using a specially designed optical microscope which, unlike current electron microscopes, permits examination of the subject tissue in a live setting. The microscope was able to image live bacteria and viruses. To this day, the ability to replicate the Rife microscope is yet to surface again.

Rife's findings remain significant. His so called “Universal Microscope” developed in 1933 was named because of its adaptability in all fields of microscopy, being fully equipped with separate substage condenser units for transmitted and monochromatic beam, dark-field, polarized, and slit-ultra illumination, including also a special device for crystallography. The entire optical system of lenses and prisms as well as the illuminating units were made of block-crystal quartz which is transparent to ultraviolet radiations.

Disposed between the source of light and the specimen were crystal quartz prisms which were used for the purpose of polarizing the light passing through the specimen. Polarization is important in that light waves vibrate in all planes perpendicular to the direction in which they are propagated.

Quartz prisms on the universal microscope were rotated with vernier control through 360 degrees, and could bend the transmitted beams of light at variable angles of incidence. When a portion of the spectrum was reached in which both the organism and color band vibrate in exact accord, a definite characteristic spectrum was emitted by the organism.

For example, bacillus typhosus emitted a blue spectrum with a polarization of +4.8 degrees. The predominating chemical constituents of the organism were ascertained after which the quartz prisms were set, by means of the vernier control, to minus (−) 4.8 degrees so that the opposite angle of refraction may be obtained.

The specimen was illuminated with a beam of monochromatic light at an identical frequency to that of the organism which enabled viewing of the organism stained in its true chemical color. It was thought that the Universal Microscope would permit detection of disease organisms such as those of tuberculosis, cancer, sarcoma, streptococcus, typhoid, staphylococcus, leprosy, hoof and mouth disease, and others and they would be eradicated when exposed to certain lethal frequencies, coordinated with the particular frequencies peculiar to each individual organism, and directed upon them by rays covering a wide range of waves.

Rife designed and had constructed an oscillator which was capable of generating a wide spectrum of individual frequencies. Even with this technology, there was no quick and easy way to determine the frequencies of a particular living organism.

It was a painstaking process of examining the culture while stepping through a wide array of frequencies, wherein Rife found one which “devitalized” it. Rife would often sit for 48 hours before the microscope isolating a specific frequency which he called the Mortal Oscillatory Rate, or MOR for many disease organisms.

In some cases, these germs or bacilli would literally shatter or explode under the influence of his frequency instrument. In other cases, the forms would remain unchanged in appearance, but would no longer be motile, and would not produce disease. Thus he used the term “devitalized” rather than “destroyed” in these instances. Recent work has shown many of these ultra-microscopic forms to be extremely rigid and resistant to deformation.

Rife isolated the organisms and found an MOR for tuberculosis, e-coli, tetanus, chickenpox, herpes type virus, pin worms, streptothrix (fungi), rabies and altogether over a forty year period, the MOR for about 600 different forms of bacterial and viral forms. The primary frequencies used ranged from the low audio up as high as the limit of short-wave, with several frequencies being combined, and acting both as a carrier as well as a treatment frequency. Rife over-modulated these so as to produce a pulsed wave-form which acted on the organisms in the same way as a tone can shatter a glass when vibrated at the glass' resonant frequency. Rife and his associates found that the human cell was hundreds of times more resilient than the cell walls of disease organisms, and there was never any observed ill effect from immersion in any of these waves.

Rife's Proofs of Pleomorphism

Rife stated that he and his colleagues had narrowed the actual distinct number of groups of pathogenic bacteria to 10. In his 1953 book, Rife commented on this: “We have classified the entire category of pathogenic bacteria into 10 individual groups. Any organism within its group can be readily changed to any other organism within the ten groups depending upon the media with which it is fed and grown. For example, with a pure culture of bacillus coli, by altering the media as little as two parts per million by volume, we can change that micro-organism in 36 hours to a bacillus typhosis showing every known laboratory test even to the Widal reaction. Further, controlled alterations of the media will end up with the virus of poliomyelitis or tuberculosis or cancer as desired, and then, if you please, alter the media again and change the micro-organism back to bacillus coli.” It can be seen that Rife had discovered something that was contrary to the state-of-the-art knowledge of virology.

Rife contended certain conclusions escaped earlier researchers simply because they lacked the evidence of their eyes in seeing these forms develop from a single entity: pleomorphism. They require optical microscopes with a power of magnification and resolution beyond the typical 2,000 power instrument. Beyond even the theoritical wavelength limit of optical instruments.

Rife's work suggested that the wide array of disease bacterium were merely differentiation phases in a life-cyle of an as of yet undetermined entity. Researcher Gaston Naessens has verified many of Rife's findings, and has delineated 16 phases of change of what Rife called the premodal identity or body, which Naessens calls “somatids”. Despite Rife's proofs of so-called pleomorphism, and the work of others along that line today it remains a controversial issue.

Rife soon discovered that he could culture something he called Bacillus X or BX virus from every instance of Carcinoma. He could subsequently inoculate rats with this BX virus and spontaneously cause the formation of solid malignant tumors. He also discovered that by irradiating the inoculated animals with his “frequency instrument”, “Beam” or “Ray Tube” he could cause the tumors to be destroyed while leaving healthy tissue intact. In countless studies, Rife cured all the rats in which the BX induced cancer using the “beam”. (He claimed another form, which he named Bacillus Y or BY was found in every instance of sarcoma). It is claimed that Rife's ray tube was effective against human cancer through a clinical study done under the auspices of the University of Southern California. However USC disputes this, saying no records exist that any such study ever took place. Nevertheless, according to John Crane, a special Medical Research Committee had been committed to looking into the work of Rife in 1934. A Dr. Milbank Johnson is said to have supervised the committee. They found 16 terminally ill patients, all suffering from late-stage cancer, and brought them down to a ranch which had been owned by a member of the Scripps family, of the Scripps Oceanographic Institute fame. Rife described the treatment in 1934:

“With the frequency instrument treatment, no tissue is destroyed, no pain is felt, no noise is audible, and no sensation is noticed. A tube lights up and 3 minutes later the treatment is completed. The virus or bacteria is destroyed and the body then recovers itself naturally from the toxic effect of the virus or bacteria. Several diseases may be treated simultaneously.”

The first clinical work on cancer was completed under the supervision of Milbank Johnson, M.D. which was set up under a Special Research Committee of the University of Southern California. 16 cases were treated at the clinic for many types of malignancy. After 3 months, 14 of these so-called helpless cases were signed off as clinically cured by the staff of five medical doctors and Dr. Alvin G. Foord, M.D. The treatments consisted of 3 minutes duration using the frequency instrument which was set on the mortal oscillatory rate for ‘BX’ or cancer virus (at 3 day intervals). It was found that the elapsed time between treatments attains better results than the cases treated daily. This gives the lymphatic system an opportunity to absorb and cast off the toxic condition which is produced by the devitalized dead particles of the ‘BX’ (Bacillus X) virus. No rise in body temperature was perceptible in any of these cases above normal during or after the frequency instrument treatment. No special diets were used in any of this clinical work, but it is believed that a proper diet compiled for the individual would be of benefit. In this putative 1934 clinic they claimed Rife's beam yielded a 100% cure rate.

For such a seemingly effective instrument, very little is known about Rife's ray device. It is speculated to have consisted of 1) a power supply or a large bank of car batteries with three motor generator sets to maintain direct current flow, 2) a frequency generator capable of modulating audio and radio frequency waves which were variable by controls and, 3) applicator tubes which were similar to Coolidge type x-ray tubes filled with a noble gas (or gases) which gave an emanation at varying frequencies forming the method of transmission from a right angle electrode which absorbed the current directly to grounded connections.

The specific frequency effective against the BX is given by various sources as 2127 or 2128 cycles per second. According to one source who worked with an original model, these numbers were not frequencies at all, but arbitrary dial settings, and should be read off as: 2,1,2,7, etc. Rife indicated he was using a +/−25 meter wavelength (11.78 Megacycles) as well as another wavelength which was 17 6/10 meters (17.045 Megacycles). According to principles of heterodying, these two frequencies would result in two major side bands at 28.825 Mhz, and 5.265 Mhz.

There are several aspects of Rife's original work that is not clear. Some of Rife's work using direct connection electrodes in place of the plasma tube indicated the BX was attracted to the cathode or negative terminal. Other works of Rife reported that the cancer was electro-statically bipolar and could be segregated into two groups: some attracted to the positive terminal and some to the negative terminal respectively. Samples from a segregated group could not produce the disease.

The Rife modality of treatment of disease is said by some to fall under the classification of physical remedies by many along with Roentgen or X-rays, light, diathermy or heat treatments. It is interesting to note that X-ray had no effect on the BX or BY. Rife used ionizing radiation to culture them. For this reason Rife was insistent that X-ray was to be avoided, as it had the propensity to activate or stimulate the pleomorphism and was not a treatment. This suggests some other interesting observations: first, that the BX and BY are of such dimensions that ordinary staining compounds are useless.

Although chemotherapeutic compounds are directed towards cellular processes and not virus, the same limitations apply: they won't affect ultra-entities. Rife observed that the BX-BY were anaerobic. Necrotic tissues which result from chemotherapy will have a low oxygen saturation. Further, these standard approaches to cancer treatment are useless against such a cause of cancer and create circumstances similar to those used by Rife for culturing cancer. Furthermore, hard X-ray and chemotherapeutic agents are in themselves carcinogenic due to their affect on the genome.

There remains a significant need today to resurrect Rife's technology in an improved manner. The present technology is an attempt to nullify or alleviate the pathologies by way of Chemicals, (as Chemotherapy, Drugs, Nutrients, Antibiotics etc;) Electromagnetic energy (as Microwaves, Diathermy, Light, X-Ray, Radiation, etc;). Accordingly, the present invention provides a solution to aid in the determination of MOR's and method of treating using the MOR.

### SUMMARY OF INVENTION

It is an object to detect abnormal pathologies in the human body using resonance.

It is another object to provide a resonance detection system for detecting abnormal pathologies in the human body.

It is yet another object to provide a resonant frequency treatment of an abnormally detected tissue.

Accordingly, the present invention is directed to a method and system having the ability to detect pathology in a human body, and nullify that pathology by subjecting the pathology to its own resonant frequency.

This invention is related to detecting the areas of the pathologies, finding the resonant frequency of the pathology and subjecting the pathology to its own resonant frequency at a sufficient intensity to debilitate the pathology, thus allowing the body to heal, and free itself of the effects of the pathology. This is done via the aid of a bio-physiological feedback system connected to the body, and by the resonating effects of the applied resonant frequency to the pathology.

A resonance system is provided for use on a subject having a biological pathology. The system includes a wave generator operably disposed adjacent a subject for generating a wave (which can be an electric wave) at a predetermined frequency, a resonant frequency sensor and generator operably disposed adjacent the subject for sensing a resonant frequency of a predetermined area of the subject in response to the predetermined frequency and generating a resonant frequency signal in response to said sensed resonant frequency, and a device (which is preferably computer based) operably associated with the sensor for receiving the resonant frequency signal and manipulating the resonant frequency signal in a manner to be displayed. Further, the device causes the wave generator to generate at a sufficient intensity and at a resonant frequency to debilitate the pathology. A method of detecting a resonant frequency in a subject having a biological pathology and treating the pathology using the sensed resonant frequency are also provided.

### BRIEF DESCRIPTION OF THE DRAWINGS

FIG. 1 is a chart of the dermatomes of the body that will be used in determining the area for the placement of the ECG electrodes to the device.

FIG. 2A is a graph of the reaction of that a tumor in the lungs which was subjected to a particular frequency.

FIG. 2B is a graph of the reaction of that the same tumor in the lungs exhibited to a different frequency and shows a resonant frequency.

FIG. 3A is an x-ray of the lungs prior to subjecting the tumor to a resonant frequency.

FIG. 3B is an x-ray of the lungs after subjecting the same tumor to a resonant frequency as determined in FIG. 2B.

FIG. 4 is a schematic of a system of the present invention.

### DETAILED DESCRIPTION OF THE PREFERRED EMBODIMENT

Referring now to the drawings, the coordinated resonance detection system of the present invention is generally designated by the numeral 10. There are several instruments involved in this system 10 and the method and procedures for using the same. It is a premise that all matter and particles in the universe have a resonant frequency. The device 10 is able to record and graph a resonant frequency of a particular pathology as well as the effects of different forms of medication, chemicals or nutrients on a given pathology.

This procedure used in employing the system 10 encompasses a feedback system utilizing resonant frequencies, and as a result, a brief review of the terms and descriptions of frequencies follows.

Everything in the Universe has a predetermined frequency. Frequencies are the basic forms of the Universe and these frequencies are measured in Hertz. Hertz is the number of oscillations or vibrations per second.

There are different styles of frequencies such as sine, square, spike, and triangular shaped. The present invention employs, for example, a sine wave form for its detection procedure. Frequencies are expressed in terms of oscillations per second (osc/sec).

1 Hertz=1 osc/sec

1 Kilohertz==1 Khz=1000 osc/sec (thousand)

1 Megahertz==1 Mhz=1,000,000 osc/sec (million)

1 Giga hertz=1 Ghz=1,000,000,000 osc/sec (billion)

1 Tetra Hertz=1 Thz=1,000,000,000,000 osc/sec (trillion)

In solids the frequencies are in the area of 1000 hz or so. In living tissue, a healthy cell can be in the range of 27 Thz. DNA of these living cells can be in the range of 8 Thz. All forms of matter, gas, liquids, solids, light, chemicals etc: have a frequency. Smaller objects have relatively higher frequencies than larger objects. Further, the denser matter is, the lower the frequency is exhibited.

Every object, e.g., biological entity or cell, has a resonant frequency. Multiple cells can form a larger mass such as an organ, and the organ then will have its own resonant frequency. The DNA/RNA protein fragments etc; in the envelope of the individual cell, will have its own specific resonant frequencies. The human body as a whole has its own resonant frequency depending on the size and structure of the body (height, weight, etc).

For example, a man about 5′9″ at 154 lbs would have a resonance of about 70 Mhz; a woman about 5′3″ at 135 lbs would have a resonance about 80 Mhz; and a child about 4′6″at 71 lbs would have a resonance about 95 Mhz. As previously discussed, disease such as bacteria, viruses, cancer, and any other form of pathogens that can afflict the biological entities have their own resonant frequencies which are believed significantly lower than normal tissue and within measuring capability of the present invention.

The system 10 of the present invention detects and then subjects the disease to its own resonant frequencies. This is referred to herein as “Coordinative Resonance.” Coordinative resonance is a phenomenon that occurs when a given frequency is applied to an object of a like frequency causing that object to vibrate or resonate resulting in the disruption or nullification of a disease or pathology.

The present invention can be used to treat human or animal subjects, for example. The subject used here is for example, a human body. The invention employs a “biofeedback system” to detect the resonance of a given pathology, and feedback that particular frequency to debilitate that pathology, by using the resonant frequency at a higher intensity than used for the detection of the resonant frequency.

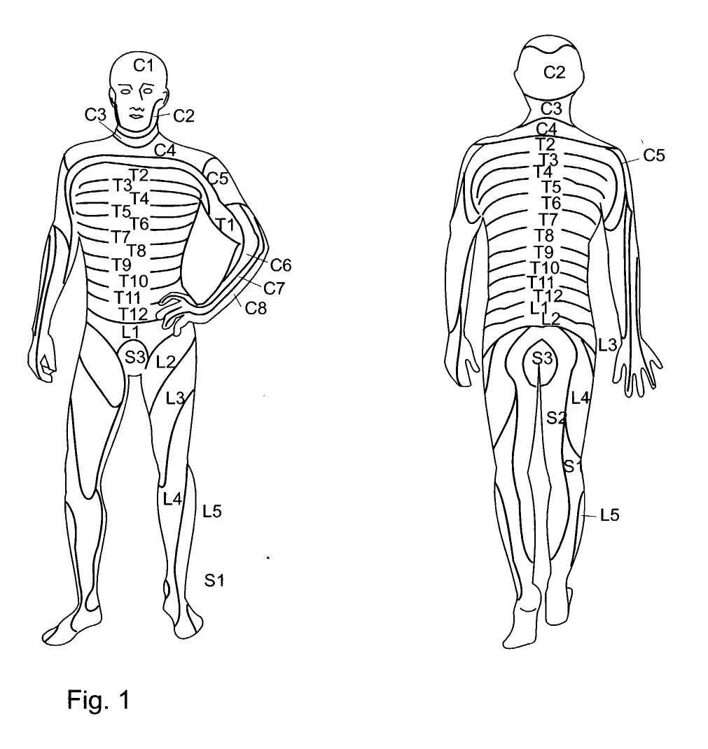

The coordinated resonance detection system 10 in one aspect includes a signal electrode 9a such as an Ag/AgCl electrode, shown on an area adjacent to the dorsal spine, but can also be on a forearm or calf of the leg, and can be operably disposed on a dermatome. FIG. 1A depicts a general representation of dermatomes, i.e., a representation of cutaneous (sensory) distributions of spinal nerves. This shows the segmental areas of the skin that carry neuron impulses toward the central nervous system (afferent) as well as neurons that carry nerve impulses away from the central nervous system (efferent).

These dermatomes enable any pathology of any parts or organs of the body to be detected. A reference electrode 9b such as an Ag/AgCl electrode is operably disposed on another extremity, preferably distal to the signal electrode 9a. The electrodes 9a are operably connected to a front end of picoammeters 15 and 71, respectively, by way of cables 11 and 13, respectively, and are generally set to measure currents in the range of 1 picoamp to 100 microamps. Cables 12 and 14 are also equipped with electrode 9b, respectively, for establishing reference electrodes. Optionally, strip electrodes 68A, 68B, 68C and 68D can be used in place or with the electrodes 9a. The strip electrode 68A can be for the cervical area, 68B for the dorsal area, 68C for the lumbar area and 68D for the sacral area, which can be used in conjunction with input cable 11 and/or 13. Alternatively, a probe electrode 7 can be used by an attendant professional skilled in a manner to determine a spike in a reading.

The picoammeter 15 is operably connected to an analog isolation amplifier 17. A shielded analog cable 19 interconnects the amplifier 17 with an A/D converter 21, such as Labjack Model U12, and an RF choke 23 is operably disposed about the cable 19 to block rf currents. A USB connector 25 interconnects the A/D converter 21 to a USB port 27 of a computer 29. The computer 29 can be a state of the art computer, such as a laptop computer, having, a processor, memory, an operating system, monitor, control and analysis software operably disposed in the memory for receiving and storing a resonant frequency signal in said memory thereto, wherein said control software which is described herein.

The computer 29 is equipped with an RS 232 port 31 to which an optical fiber modem 37 such as is available from Versa-link and is well known to one skilled in the art. Optical fiber modem 37 operably interconnects optical fiber pair 33. An RF choke 35 is similarly provided in connection of RS 232 adapter of the modem 37 to RS 232 port 31.

The Versa-link optical fiber pair 33 includes a transmit optical fiber 39 and a receive optical fiber 41. An end of the Versa-link fiber 33 includes a Versa-link optical fiber modem 43 having an RS 232 adapter which connects to a frequency synthesizer 45, which can be for example a Global Specialties Model 2003.

The frequency synthesizer 45 is operably connected to a modulator 47 which in turn is connected to an RF oscillator 49. The RF oscillator 49 is operably connected to an RF amplifier 51. A transmission line 53 connects the RF amplifier 51 with an impedance matching circuit 55 which in turn connects to a discharge or plasma tube 59 (filled with argon or helium or other suitable noble gas or mixture). The plasma tube 59 which is provided can be a 2″ diameter×18″ long, for example.

One or more picoammeters 15 and 71 can be employed, e.g., Kiethley picoammeters, capable of digital readout, + and − cable inputs, with analog outputs. The picoammeter 71 is operably connected to an analog isolation amplifier 73. A shielded analog cable 74 interconnects the amplifier 73 with an A/D converter 77, such as Labjack Model U12. An RF choke 75 is operably disposed about the cable 74 to block rf currents. The converter 77 can also be operably connected to USB connector 25.

The pair of electrodes 9a, 9b can be of the type such as ECG heart monitoring disposable electrodes. Probe 7 can be made from non-conducting material ½″ diameter and 4″ long with a ¼″ thick zinc contact surface and is also provided for use with a dermatome chart for initial detection. The probe 7 is utilized initially with the patient in locating the strongest signal obtainable from the various dermatomes. Once this strongest signal is obtained, the position is marked and an ECG electrode 9a, for example, is placed at this point.

In one instance a frequency counter function is provided by the computer 29 via its analysis and control software. The analysis and control software performs the following functions.

1. The control software scans from a start frequency to a stop frequency and communicates this frequency value to the frequency synthesizer 45 (function generator—such as the Global Specialties Model 2003) via the optical fiber modems 43.

2. As each frequency is transmitted, the software provides a dwell time (how long the frequency is transmitted before changing to another frequency) in the range of 50 milliseconds to ¼ second (250 milliseconds).

3. As each frequency is transmitted, three channels of the A/D converter 21 (LabJack U12) are sampled with the sample readings stored on the computer's 29 (laptop) hard disk for later retrieval and analysis.

4. By way of example, there are 10,000 samples taken in each full spectral scan (from the start to stop frequency).

5. The preferred mode currently employs three user selectable scan ranges: 1 hz to 10,000 hz (to accommodate the older function generators), 1 hz to 100,000 hz and 1 hz to 1.0 Mhz. It is contemplated that the software can include frequencies from 1 to 10 or even 20 Mhz. Increasing the frequency range requires either additional time for the scan or a reduced resolution of the scanning frequency.

6. The control software enables storage, retrieval and playback of all recorded data. Currently, the data collection consists of one or more channels from one or more separate picoammeters/amplifiers.

7. A visually displayed start button, a stop button and a pause button are built into the software to enable full control of the scanning function.

8. After the scan is complete, the control software allows the user to look at a time-expanded view of the recorded data in ten (10) windows visually displayed on the screen of the computer 29 consisting of the primary scan interval divided into ten (10) equal range segments. The user can then use a mouse curser to point to a particular location of interest in the data and click on it. This action allows the user to determine and select the frequency responsible for producing the salient data point. The software allows the user to store the frequency data for this data point in an array for later use.

9. The array defined by way of example in the software has the capability of storing up to 120 said data points. Each of the 120 data points can also store a therapy duration time for use later in treating a particular pathology.

10. The 120 data points with the commensurate therapy time durations make up a therapy table which can be stored under a patient name, or other common name for later retrieval.

11. The control software can subsequently run through a therapy table and provide therapy to the patient once the scanning is complete.

12. Alternatively, the therapy table can be downloaded to computer based device (which can be a small portable cpu) which can be accessed later to provide therapy at a later time as needed by the patient.

13. The software is equipped through the above described system 10 for measuring current at particular dermatomes and to enable the operator to choose the dermatome that has the strongest physiological response to the therapy stimulus.

14. The software is capable of manipulating (editing) the data as needed manually once the scanning and data point selection have been completed.

In addition to the computer 29, a conventional frequency counter 61 can be provided which can be operatively connected to the frequency synthesizer 45 to record the observed frequencies. The picoammeter 15 and frequency counter 61 readouts can be stacked one on top of the other and visually displayed by use of the control software and display screen of the computer 29.

In addition, a video camcorder 63, which can be tripod mounted, can be utilized to record the subject's physical condition and response. Preferably, the camcorder 63 can be digital and readily enable the transfer and storage of images of the subject individual to the computer 29 via a communications port 65. The subject individual information can be stored in a database of the computer 29, wherein the name, condition treated, pathology frequencies detected and treatment frequencies, voltage amount and time period treated are stored. The information data can be viewed on the display screen of the computer 29.

A method of detection and treatment of provides for the subject individual to be seated in a comfortable and lightweight chair (which is preferably easily cleaned for subsequent use) and one which does not interfere with the detection and treatment of resonant frequencies applied to the subject. The subject would sit for a period of time in front of the plasma tube 59, typically about 2½ or 3 feet away from the tube 59.

The connections to picoammeter 15 are shielded from the plasma tube 59. For example, electrical connections of about 5′ in length interconnect the picoammeter 15 to the patient and can have adapters on the ends thereof to accept either alligator type clips or a plug for the zinc probe or electrodes 9a, 9b.

As mentioned previously, the zinc probe 7 can be moved slowly down the spine to show a high reading in the dermatome area of which has the pathology. A reading can also be read on any area that the same dermatome segment reaches. Using the spinal area of the particular dermatome is believed a simple and reliable method (this is the area that the nerves enter and leave the spinal cord). The probe 7 is moved along the spine to locate an abnormal resonant reading over nerve areas of a dermatome which leads to a pathology. To probe the spinal area, it may be desirable to have the subject seated on a stool or computer type of swivel chair with the back of the chair to the side of the subject, so the spinal area to be probed is clear of any obstruction.

When probing is complete, and the electrodes 9a and 9b are in place, the subject is seated in the chair, and the electrode leads 9a, 9b are connected to the picoammeter 15. The lead from the dermatome connects to the positive cable of the picoammeter 15 (thus forming the signal electrode). The lead from the distal reference is connected to the negative cable of the picoammeter. An initial scan can be performed from 1 Hz to 7 Mhz, for example, which is a suspected range for abnormal pathology. This takes approximately 30-40 minutes using the function generator 45. The time it takes for any subsequent scan can be set for the same amount of time, e.g., 16.7 minutes (1002 seconds).

The range of frequencies scanned will determine the amount of time it will take for any given frequency detection test. For example, if a scan is made from 1 Hz to 1 Khz in the set time frame, the amount of time for each frequency is approximately 1 Hz per second. The maximum amount of frequencies that the frequency counter 22 can handle in the above time frame may be 1000, but other counters may handle higher amounts. Because of this, each group of frequencies can only be in increments of 1000, as 1-1000 Hz, 1000 Hz to 2000 Hz, and so on. These groups take a predetermined scan-time, e.g., 16.7 minutes each. Each scan interval contributes to the overall time needed to complete the full spectrum of frequencies.

In the case of the first scan, there may be 1 or 2 peaks to be rescanned, in the second scan there may be 5 peaks to be rescanned, and the third scan may have 10 or so peaks to rescan for resonance, etc. The function synthesizer 45 modulates the carrier frequency of the linear amplifier 51 (e.g., 14.318 MHz), with the frequency generated, which is transferred to the plasma tube 59 which then radiates the frequency to the subject.

The carrier frequency (radio frequency) is the penetrating frequency. The current readings of the picoammeter 15 may jump when the plasma tube 59 is first turned on, but will normalize to a level reading in a few seconds before starting the scan sessions.

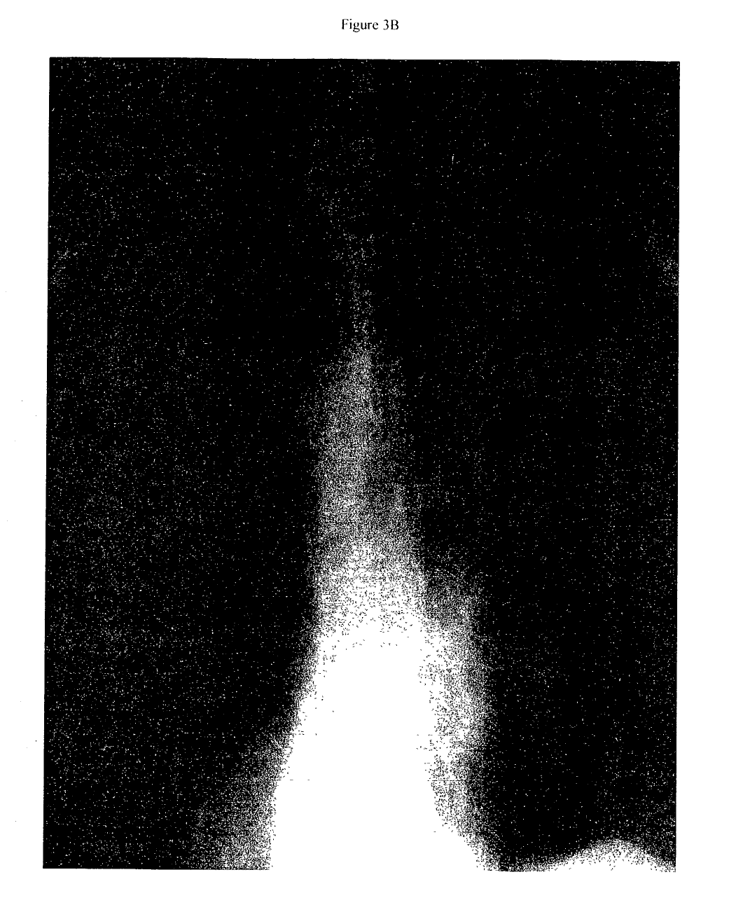

The preceding applications were for the coordinative resonance detection of a biologic pathology. In one case study, a tumor was detected and treated. FIGS. 3A and 3B depict the before and after treatment pictures using the present invention. (An elderly female suffering from adenocarcinoma of the lungs as a result of long-term cigarette smoking):

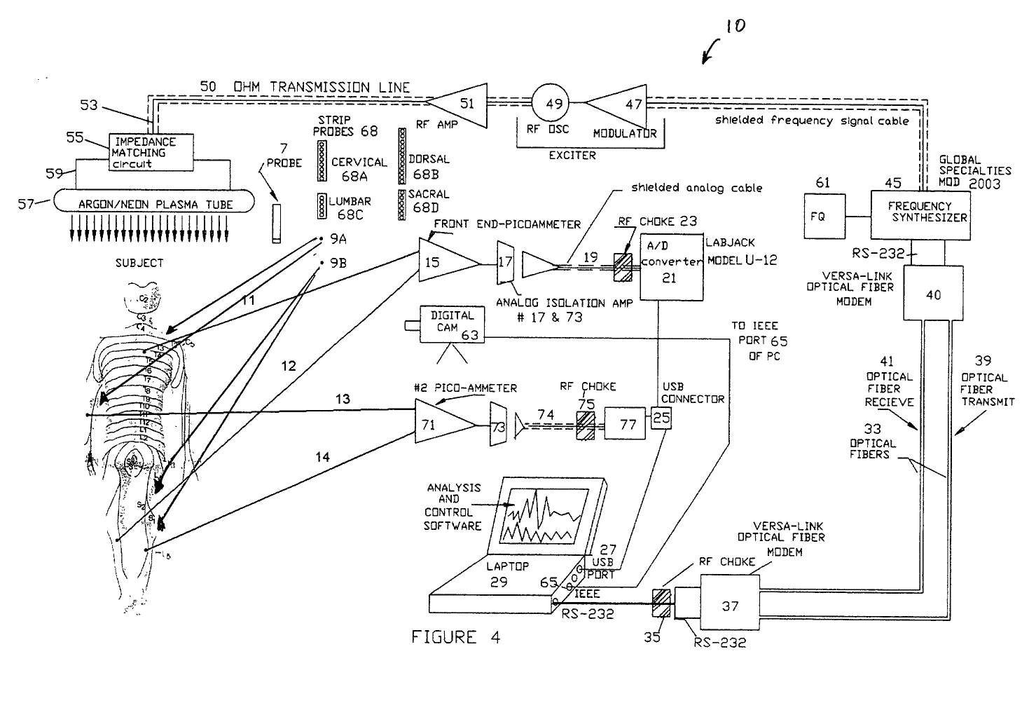

The system 10 also enables feedback detection of the effects of chemicals, such as the different types of chemotherapy, as well as all types of pharmaceuticals, vitamins, minerals, nutrients, etc., and their effects on the various pathologies as tumors or cancers, bacterial, viral, or other forms of diseases, that afflict the human or animal species, which can be easily be recorded and or graphed. This feedback detection can be used with some of the procedures used previously as well as the addition of additional electrodes described above on the opposing areas as the calf of the leg, and the forearm as shown in FIG. 4.

These additional electrodes can be connected to a second picoammeter 71, and enable the recording, storage, and display of the resonant effect of the chemical. In this case, no resonances are determined. Instead, a deviation of baseline readings of the picoammeters 15 and 71 resulting from the exposure of the subject to the chemicals or nutrients are observed. This helps determine whether the chemicals or nutrients are of any value as to their affects on the pathologies or on the body generally. The results will also show a change in the base line readings of the body and the base line readings of the pathology, if the chemicals or nutrients are going to be effective. This will encompass a lot of readings, depending on the number of chemicals or nutrients used.

It is also contemplated by the present invention that when the chemicals or nutrients that appear to be the most effective, then a spectroscopic or spectrographic analysis will determine their specific frequency, and then the chemicals' or nutrients' frequencies can be matched to the resonant frequency of the pathology as determined previously, by the coordinative feedback system 10, and then taken as a follow-up regimen after the resonant therapy has debilitated the pathology. This follow-up regimen of the chemicals or nutrients with the same resonant frequencies determined to be effective, can then be given to the subject to minimize or prevent any possible recurrences in the same or remote areas of the subject in which the pathologies have been debilitated. As time progresses, charts of chemicals and nutrients can be made up through spectral analysis and catalogued for quick references to the above procedures.

Since this patient's tumor or pathology was located in the lungs, as mentioned herein above, the dermatomes for the lungs were found in the dorsal or thoracic (chest) area. These were easily accessed over the spinal nerve areas (each side of the spinous process of the vertebrae of the spine in the chest area), and the therapeutic procedure was as follows.

A picoammeter 15 was connected by way of the electrodes 9a and 9b to the body. The neutral or minus electrode 9b can be attached to the left or right forearm or calf of the left or right leg.

To probe the right side of the spinal area, the electrodes are placed on the left forearm or preferably left calf of the leg. The areas to which the electrodes are attached are first swabbed with alcohol and dried prior to adhering the electrodes. The reason for spacing the electrodes a sufficient distance is to minimize any possible interaction between the electrodes, by increasing the impedance.

The zinc probe 7 was connected to the plus lead of the picoammeter 15. The picoammeter 15 was set to the Microamp range. The zinc probe 7 was subsequently placed on either side of the spine of the bony protuberances of the vertebrae, from the base of the neck to about the bottom of the rib cage.

Using sufficient pressure to slightly depress the skin, the probe 7 was slowly moved down the spine. With a water soluble marker (preferably red), the area of the highest reading was marked (on top of the bony prominance of the vertebrae adjacent to the area being read). This permitted an area to be cleaned with an alcohol swab before applying the ECG electrode 9a and without losing placement position.

The baseline reading was noted, and the picoammeter 15 was set to a range that would allow for spikes in the readings (usually 100 Microampere range) or lower if only one digit is displayed. It may be necessary to switch to the Nanoamp settings in the case of weak readings with some subjects. Optionally, an auto-ranging picoammeter can be employed to perform the above function.

The camcorder 63 (which was set to record the meter and frequency counter readings), and computer 29 were utilized at this point for recording purpose. The range of frequencies to be scanned must be within the range of the function synthesizer 45 and plasma tube 59. A spectral scan was performed over the range of frequencies with the plasma tube 59 and the subsequent measured biocurrents were recorded.

After completion of the scan, the recorded readings were analyzed. The peak measurements from the picoammeter 15 were selected and the associated frequencies recorded as data points. The computer 29 via analysis and control software provided display and printouts of line or bar graphs, for example, of the readings showing the peaks and dips of the meter readings along with the frequencies.

The scan process was repeated for the peak readings only, and in the case of the peaks being relatively few, the frequencies were applied one at a time for about 15-20 seconds waiting 10-15 seconds before applying the next frequency (this is done so that if the pathology resonates, it will do so within a few seconds after the plasma tube 59 is shut off and then the resonance can be recorded before engaging the next frequency.)

In another subject case study, recorded pathological resonances lasting as long as 45 minutes were observed and had continued resonation even after removal of the stimulus. This observation is consistent with the case study where the patient suffered from large tumors occupying about 60% of the lungs.

A table of resonant frequencies were determined for use in debilitating the pathology. It is recognized that in some cases where metastasis has involved many areas of the body, (bones as well as soft tissue), there may be several resonances to be recorded and used. Four or more different resonances have been observed in the same subject.

After the resonance points were determined, candidate frequencies were applied to the subject at a higher wattage than used in the scanning mode to debilitate the pathology. For example, if 15-25 watts have been used to determine resonances, then 35-125 watts or more can be used to debilitate the pathology.

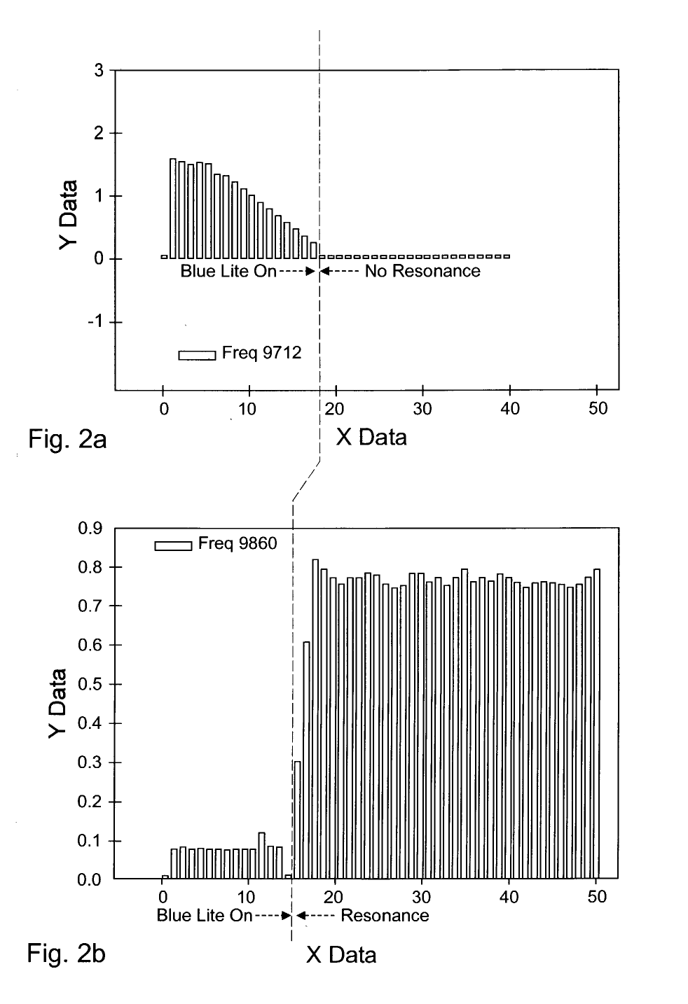

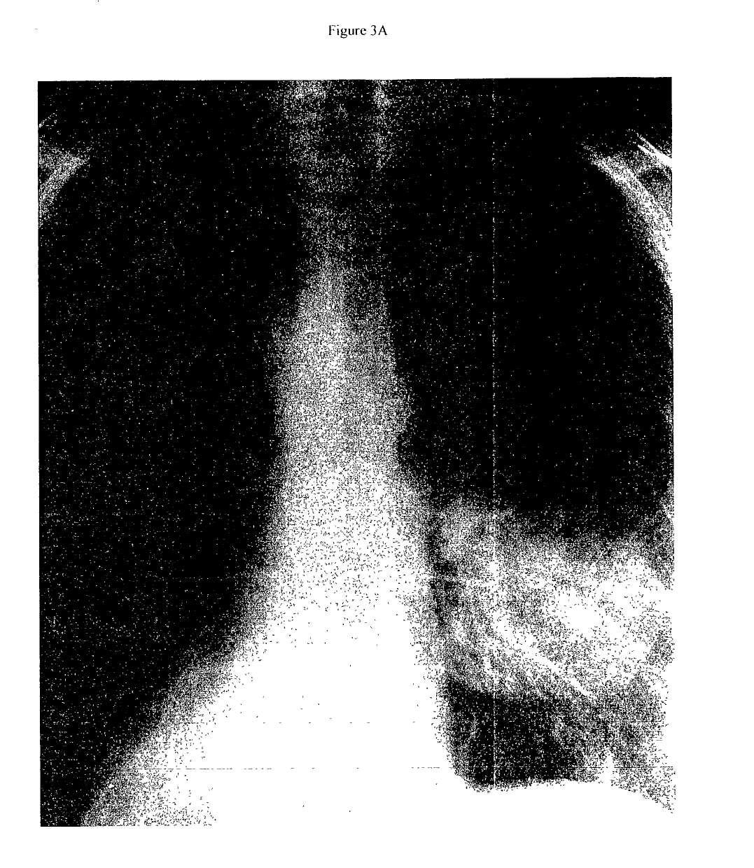

FIG. 2A shows a graph of a spike in a scan of the 9 khz range, specifically at 9712 Hz. The plasma tube 59 was on for 15 seconds and off for the remainder of the 40 second time frame, wherein no resonance was exhibited. FIG. 2B shows a graph of a spike in a scan of the 9 khz range, specifically at 9860 Hz. The plasma tube 59 light was on for 15 seconds and off for the remainder of the 50 second time frame and resonance is achieved. The time frame can be varied. It can be seen that the spike of 9712 Hz is over 1 microamp on the Y axis of the graph which is a strong spike. The Y axis represents units of amperes (function of force of the current flow). The X data represents units of Time in seconds. A strong spike is not necessarily an indication of resonance, as some would probably think.

FIG. 3A is a copy of the chest X-ray, showing the tumor after several months of Chemotherapy, prior to the scans and determination of the resonant frequency. FIG. 3B is a copy of the chest X-ray after about 12 weeks of low wattage exposure of the tumor to the resonant frequency from the plasma beam tube 59 at about 25 to 35 Watts. The use of the lower power exposures in a time frame of 3-4 times weekly was dictated by the size of the tumor. This method keeps the toxins from the debilitating of the pathology, to a level that the body can eliminate the toxins, which can be harmful if the body is overloaded with the toxins.

A proper diet, elimination and supplementation are needed to help rebuild the body's immune system. Notice also that in this X-ray there is some fibrous and scar tissue that remains after the tumor has been debilitated which does not warrant further therapy.

The above described embodiments are set forth by way of example and are not for the purpose of limiting the present invention. It will be readily apparent to those skilled in the art that obvious modifications, derivations and variations can be made to the embodiments without departing from the scope of the invention. Accordingly, the claims appended hereto should be read in their full scope including any such modifications, derivations and variations.

What is claimed is:

1. A resonance system for use on a subject having a biological pathology, comprising:
means operably disposed adjacent a subject for generating a wave at a predetermined frequency;
means operably disposed adjacent the subject for sensing a resonant frequency of a predetermined area of the subject in response to the predetermined frequency and generating a resonant frequency signal in response to said sensed resonant frequency; and
means operably associated with said sensing means for receiving said resonant frequency signal and manipulating said resonant frequency signal in a manner to be displayed.

2. The resonance system of claim 1, which further includes means for comparing said predetermined frequency signal with said resonant frequency signal.

3. The resonance system of claim 2, which further includes means for adjusting strength of said wave of said predetermined frequency and setting predetermined frequency to said resonant frequency of said predetermined area to affect a treatment on the pathology.

4. The resonance system of claim 3, wherein said comparing means includes said non-resonant frequency signal with said resonant frequency signal in order to determine said predetermined frequency.

5. The resonance system of claim 1, which further includes video capture means for visually recording characteristics of the subject.

6. The resonance system of claim 1, wherein said sensing means includes a probe.

7. The resonance system of claim 1, wherein said sensing means includes an electrode.

8. The resonance system of claim 1, wherein said sensing means includes a strip of electrodes.

9. A method of detecting a resonance of a particular biological pathology, comprising the steps of:
directing an wave of a predetermined frequency at a subject;
sensing a resonant frequency from a predetermined area of the subject in response to said predetermined frequency;
generating a resonant frequency signal in response to said sensed resonant frequency; and
receiving said resonant frequency signal and manipulating said resonant frequency signal in a manner to be displayed.

10. The method of claim 9, which is further characterized to initiate a series of frequency signals over a predetermined range of frequencies in order to determine said predetermined frequency and enable said resonant frequency to be sensed and a non-resonant frequency to be sensed.

11. The method of claim 10, which further includes comparing said predetermined frequency signal with said resonant frequency signal.

12. The method of claim 10, which further includes adjusting strength of said wave of said predetermined frequency and setting said predetermined frequency to said resonant frequency of said predetermined area to effect a treatment on the pathology.

13. The method of claim 12, which further includes comparing said non-resonant frequency signal with said resonant frequency signal in order to determine said predetermined frequency signal.

14. The method of claim 13, which further includes providing a video capturing device for visually recording characteristics of the subject.
